(19) United States
(12) Patent Application Publication
(10) Pub. No.: US 2024/0009027 A1
(43) Pub. Date: Jan. 11, 2024

(54) A WEARABLE COOLING APPARATUS FOR ON-SITE TREATMENT OF HEAT ILLNESS

(71) Applicant: CRYOGENX LIMITED, Central Research Laboratory, Hayes, Middlesex (GB)

(72) Inventor: Matt Anderson, Hayes, Middlesex (GB)

(21) Appl. No.: 18/254,095

(22) PCT Filed: Nov. 23, 2021
(86) PCT No.: PCT/GB2021/053027

§ 371 Date: May 23, 2023

(30) Foreign Application Priority Data
Nov. 23, 2020 (GB) 2018373.7

(51) Int. Cl. A61F 7/02

(52) U.S. Cl. CPC A61F 7/02; A61F 2007/0057

(57) ABSTRACT

A wearable cooling apparatus (2) comprising a pad (4), an inlet valve (6), a channel (8), and a thermally conductive membrane (10). The channel (8) is an open-sided recess formed in the surface of the pad (4) and the pad (4) is secured to the membrane (10) such that the open side of the channel (8) is covered by the membrane (10). The covered channel (8) forms a conduit (11) arranged between the pad (4) and the membrane (10), wherein the walls of the conduit (11) comprise the surface of the pad (4) and the surface of the membrane (10) and the inlet valve (6) is arranged in fluid communication with the conduit (11).

The present invention relates to a cooling apparatus for on-site treatment of heat related illness and more particularly, but not exclusively, to a compact, portable, lightweight, on-demand wearable cooling apparatus able to be deployed in multiple scenarios for the immediate treatment of heat illness on-site and in-situ. It is also envisaged that the apparatus could also be used for general therapeutic and healthcare applications, where whole body cooling would be beneficial and particularly, but not exclusively, in environments where large, non-portable, whole body cooling apparatus would be difficult to provide.

Heat stress and heat related illness caused by heat stress significantly affects the capability of numerous sectors worldwide including, for example, defence, labour industries, the sports industry and healthcare.

Though significant, heat illness is, however, a largely preventable source of illness, injury and mortality with mostly avoidable rehabilitation requirements. The majority of incidents among British defence personnel occur during physical training, with particular risk on short trial, high intensity exercises which can render personnel in critical conditions within very short time frames. Exposure to heat stress is more commonly encountered in land based exertional activity such as during fitness assessments and rigorous training. However, maritime units afloat conducting arduous activity such as fire-fighting drills, through to deployed construction tasks and loading at airbases in high thermal stress environments, demonstrate a clear need across all three services.

Heat illness constitutes a spectrum of illnesses caused by a combination of environmental load and metabolic heat production. Heat illness occurs when the body's thermoregulatory system becomes overwhelmed due to an inability to effectively dissipate heat away from the body. The condition is pathophysiology complex with incompletely understood mechanisms. The onset, symptoms and severity varies significantly to circumstances and the individual's susceptibility to heat illness, which is influenced by numerous, often unknown factors.

What is widely known, however, is that heat illness is a medical emergency and can cause permanent injury and death within hours if not rapidly treated via active cooling measures to reduce core temperature.

Varying psychological and physiological tolerances to heat mean that a single set of guidelines or procedures is insufficient to ensure the safety of all. Psychological barriers add another layer of complexity to diagnosis and treatment and ‘soldiering on’ through mild heat illness can be extremely dangerous and lead to life threatening conditions. This is mostly applicable to defence personnel who are pushed to extremely high levels of exertion in training and assessment.

Severe heat illness, often characterised as exertional heat stroke, is a medical emergency and can lead to permanent debilitating injury and death within hours. There is significant risk of both short and long term complications, particularly if there are delays in treatment. The severity and reversibility of heat injury is directly influenced by the intensity and duration of elevated core temperate. Rapid response via aggressive on-site cooling is critical to reduce the severity of injury and subsequent primary care and rehabilitation requirements.

The significant impact on life and health as a result of heat illness sustained in the UK and overseas is nearly all preventable, provided the risk factors are sufficiently assessed and appropriately managed. Appropriate management of heat illness involves the capability to rapidly reduce core temperature through active cooling techniques.

The most effective treatment method to reduce core temperature is considered to be full body ice water immersion (IWI) which is capable of delivering cooling rates of 0.35 C/min (Journal of Athletic Training). 30 minutes of full body IWI at 2 C requires approximately 300 L of water and 100 L of ice per person with additional requirements around storage and transportation. Although IWI is an extremely effective method, the volume of resources and level of logistical planning renders it impossible to implement in the vast majority of scenarios.

A more feasible, but considerably less effective and less reliable method of treatment is evaporative cooling. Evaporative cooling comprises spraying or sponging the skin with cool water and fanning. Evaporative methods have reported cooling rates between 0.035 C/min and 0.175 C/min (Journal of Athletic Training). This encompasses the 0.15 C/min minimum viable cooling rate for effective treatment, as stated by multiple national medical journals. The majority of evaporative cooling methods do not pass the 0.15 C/min minimum viable cooling rate. As with IWI, evaporative cooling has limitations to the environmental conditions in which it can be effectively deployed. When humidity is high for example, evaporative heat loss is severely diminished, making the process far less effective.

The relative ineffectiveness of evaporative cooling compared to IWI inevitably leads to increased severity of heat illness cases, higher instances of evacuation to medical care facilities and longer rehabilitation requirements. In some scenarios where resources are lacking or unavailable, ineffective and improvisational cooling methods are utilised, which can have significant repercussions regarding patient outcome.

There are very few known portable cooling products used for field treatment of heat illness. For example, as detailed in Section 1.4, JSP 539 v3.1 of the Ministry of Defence Heat Illness and Cold Injury Medical Management guidance, the use of evaporative cooling (spray and fan) is recommended, which, as discussed above, is a substantially less effective method compared to IWI. Evaporative cooling is reliant on a supply of water and the ability to generate convection currents over the individual. Additionally, evaporative cooling has limitations to the environmental conditions in which it can be effectively deployed. When humidity is high, evaporative heat loss is severely diminished, making the process far less effective. Furthermore, this technique is nearly impossible to administer during initial CASEVAC of a casualty (evacuation of casualties by air). Due to the varying environmental conditions and availability of resources, the quality of treatment greatly varies.

Targeted temperature management technology has been used in clinical environments for management of hyperthermic patients, for example as disclosed in US2019151141A1 which discloses a thermal treatment device administered in a hospital or similar environment wherein pads wrap around a patient's body and a fluid or compressed gas is passed such that heat can be taken away from the patient to cool them. It is entirely infeasible to deploy such an apparatus outside hospital care parameters.

A known portable and wearable integrated cooling system for treating heat illness is disclosed in US2020281284. This describes a portable, wearable, cooling device, wherein a cooling fluid is pumped around the garment via tubing and a pump. The cooling fluid is provided in removable and replaceable cartridges and the pump drives the cooling fluid (for example water) through a circuit of tubes into bladders that provide a cooling effect to the wearer.

The apparatus is designed to be worn whilst performing strenuous activity “in order to prevent the onset of hyperthermia” and so is lightweight and portable and enables the wearer to still have freedom of movement. The replaceable cartridges of this apparatus can be liquid or gel-based, or filled with compressed gas that cools when expanded.

Another known wearable apparatus is the CAERvest®, an endothermic hypothermic device for core body cooling.

The CAERvest® is single use product with a maximum cooling capacity of one hour and costs approximately GBP500 per unit. The product has been supplied to numerous high profile clients across multiple markets including the US Military, the London Marathon, London Air Ambulance, and multiple construction companies in the US, Australia, Singapore and Arab states. The CAERvest® has numerous critical shortcomings which limit its effectiveness and potential use cases. Critically, for example, CAERvest®'s cooling capacity is insufficiently effective to impact the majority of cases.

During a 2014 pre-clinical trial testing the cooling rates on normothermic individuals, CAERvest® produced a cooling rate 0.01 C/min. This is ten times lower than the required minimum cooling rate for heat illness. Furthermore, all participants were male. There are serious questions regarding the efficacy of the product with use on women. Poor conformability around breast tissue will substantially reduce skin surface area coverage and subsequently cooling capacity. In 2015, CAERvest® was used on a hyperthermic male individual during the London Marathon and produced cooling rates of 0.1 C/min, substantially less effective than IWI among other methods. In this case other cooling therapies had already been used to initiate cooling, further questioning the performance capacity of CAERvest®.

Additionally, due to the design of the device, the effectiveness of the cooling will vary depending on the user's somatotype and body shape. The ribbed structure reduces contact with the skin, limiting conduction, and therefore heat dissipation.

Although the product offers an adequate cooling solution in some circumstances, its lack of cooling capacity combined with inefficacious product features leads to a device that has inconsistent performance and limited use case scenarios.

The present invention addresses all the shortcomings of the CAERvest® and the other known prior art. The present invention is designed to be operable on numerous body types and is equally effective for both men and women. The cooling duration of the present invention is also not limited to an hour. As will be discussed in detail subsequently, the present invention uses pads, a single set of which can deliver consistent levels of cooling if sufficient gas supply is available.

It is an object of the present invention to provide a wearable and portable rapid cooling apparatus for the in-field treatment of heat illness which overcomes the failings, as discussed, of current treatments.

According to the present invention there is provided a wearable cooling apparatus as defined in the appended independent apparatus claim. The walls of the conduit are formed by both the membrane and the pad. As such, fluid injected along the conduit conductively interacts with both the pad and the membrane simultaneously.

Further preferable features of the apparatus of the present invention are defined in the appended dependent apparatus claims. For example, the channel could be formed in the surface of just the membrane (rather than the pad) with the open-side of the channel covered by the pad (rather than the membrane). Such an arrangement would have the same effect of enabling fluid injected along the conduit to conductively interact with both the pad and the membrane simultaneously.

According to a further aspect of the present invention there is also provided a method of manufacturing said apparatus as defined in an appended independent method claim. Further preferable features of this method may be defined in appended dependent method claims.

It is also possible that the apparatus of the present invention could be used to prevent a wearer from overly sweating in hot or stressful situations, or from becoming red-faced, for example when performing in public. The apparatus of the present invention is lightweight and portable enough that it could be worn beneath clothing such that its presence is concealed, disguised or completely hidden. It may be advantageous to use the apparatus of the present invention in a professional setting to appear less nervous, or to sweat less when in a highly stressful situation and sweat would be detrimental to performance.

An individual with heat illness will display various cosmetic symptoms. For example, for an individual who is perceived to be dehydrated, having an altered psychological state, being confused, dizzy, and/or having muscle twitches, etc, sweating and skin condition is difficult to define. Throughout the stages and severity of heat illness (e.g. heat syncope, heat cramps, heat exhaustion, heat stroke) the physiology of the skin changes. Typically, an individual with heat exhaustion will have cool, pale and clammy skin coupled with heavy sweating. In contrast, an individual with heat stroke will have hot, red, dry skin and no sweating will occur. Any of these aforementioned physiological aspects of a wearer of the present invention could be monitored and the cooling effect be either increased or decreased as appropriate or as preferred by the wearer.

Accordingly, according to a further aspect of the present invention there is also provided a method of effecting the cosmetic physiological response of a wearer of the apparatus according to the present invention, the method comprising the steps of, a) monitoring physiological aspects of the wearer, and b) altering the cooling effect of the apparatus according to the preference of the wearer. Further preferable features of this method are defined in appended dependent cosmetic method claims.

An embodiment of the present invention will now be described with reference to the accompanying drawings, in which:

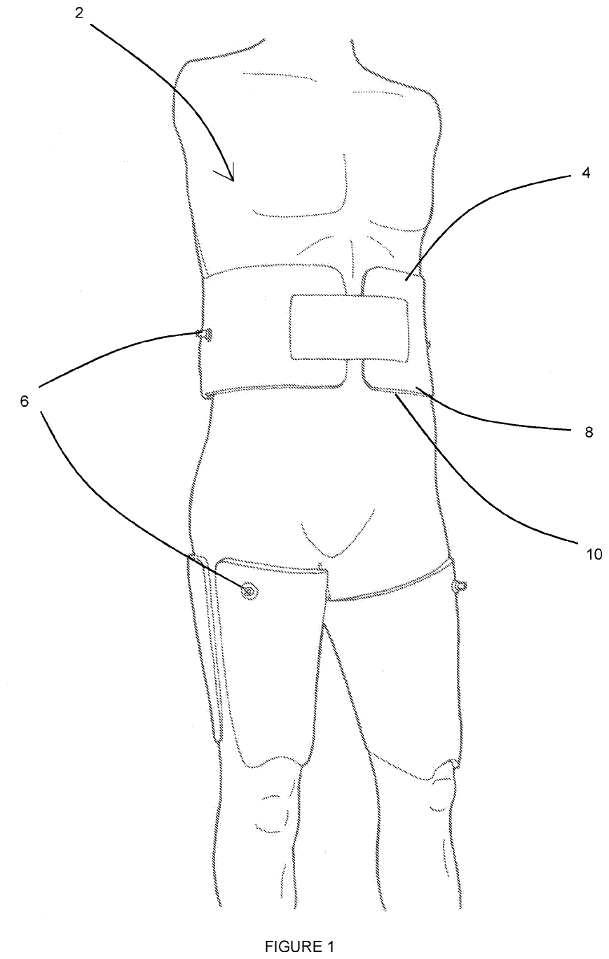

FIG. 1 is a perspective view of pads of the present invention fixed to a patient;

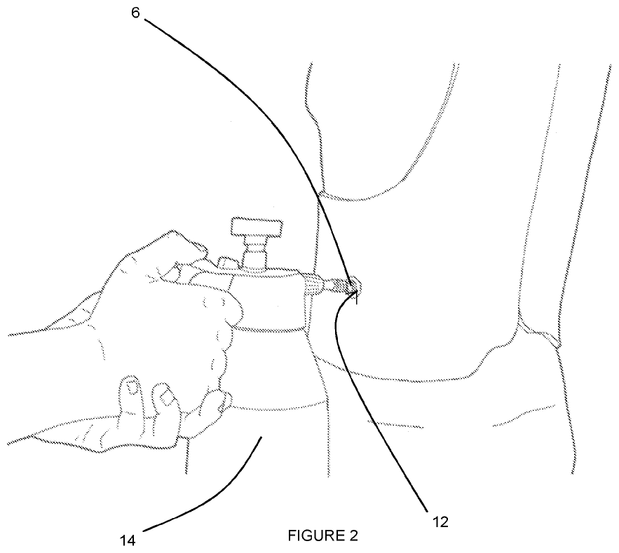

FIG. 2 is a perspective view of a part of a pad in accordance with the present invention, showing a gas canister affixed to an inlet valve such that cooling gas can be injected in said pad;

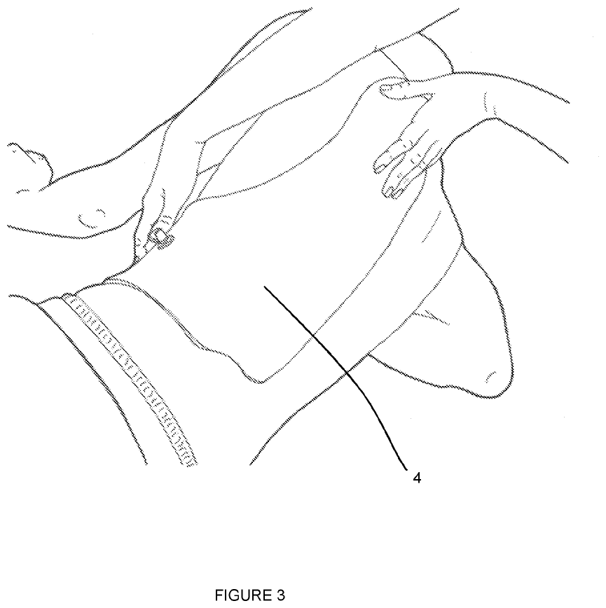

FIG. 3 shows an example of an embodiment of the pads of the present invention in use on a patient;

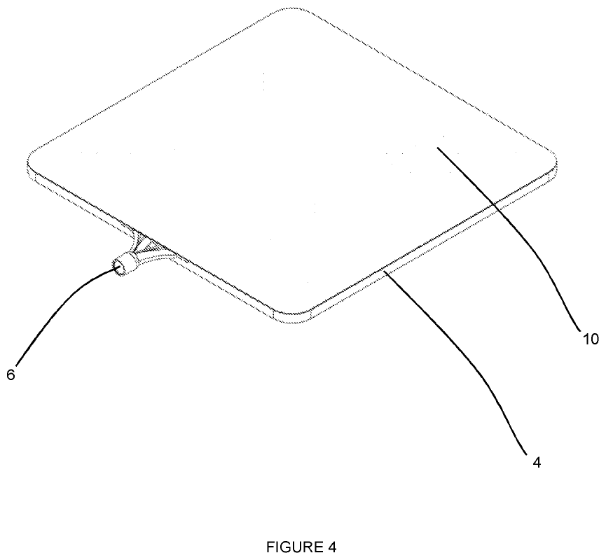

FIG. 4 is a perspective view of an embodiment of a pad of the present invention;

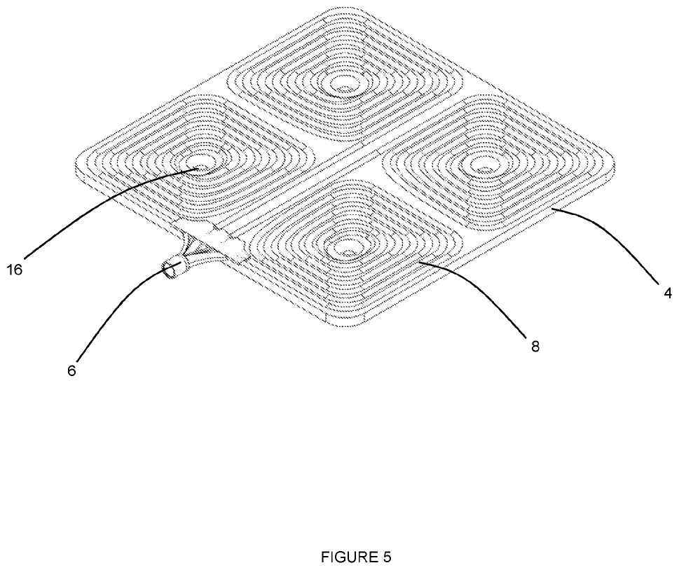

FIG. 5 is the pad of FIG. 4, with the uppermost surface removed to show the path of a network of channels moulded into said pad and the use of a 4-way splitter valve;

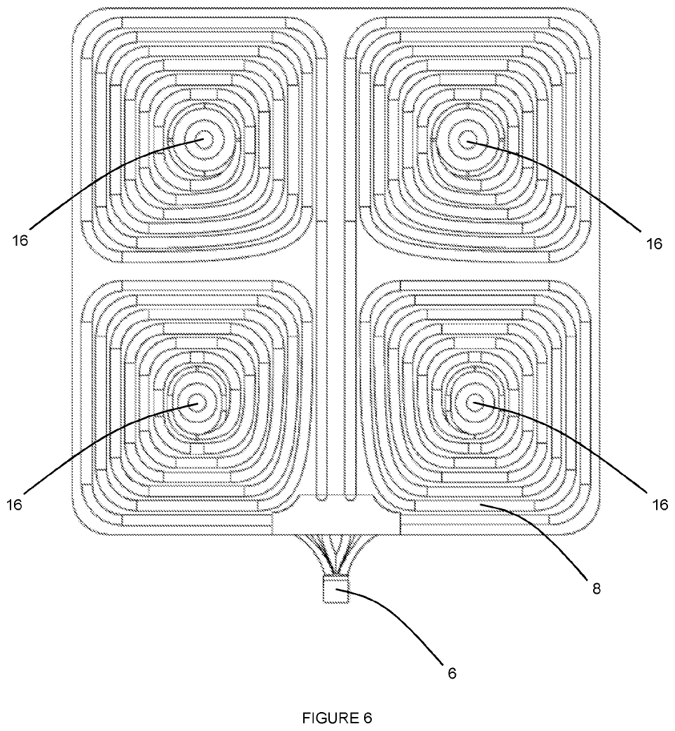

FIG. 6 is an overhead view of the pad of FIG. 5;

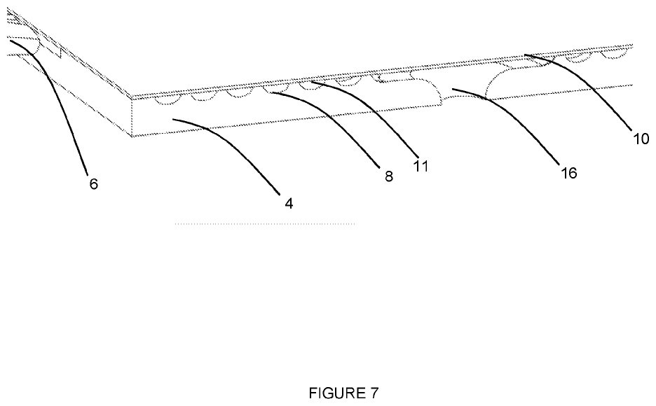

FIG. 7 is a partial perspective side view of a pad in accordance with the present invention, showing the U-shaped profile of the channels moulded to said pad and the hydrogel membrane affixed atop;

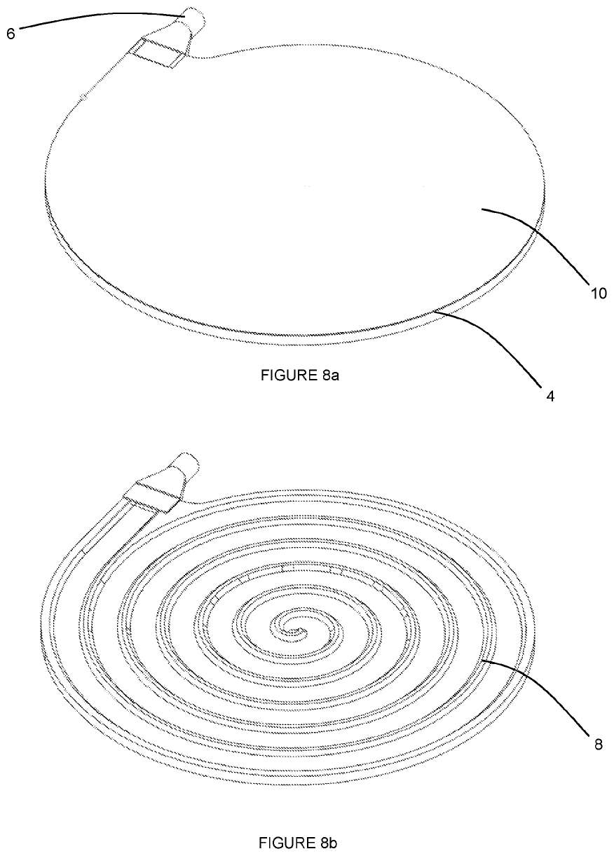

FIG. 8a is a perspective view of an alternative embodiment of the pad of the present invention;

FIG. 8b is the pad of FIG. 8a, with the uppermost surface removed to show the path of a network of channels moulded into said pad;

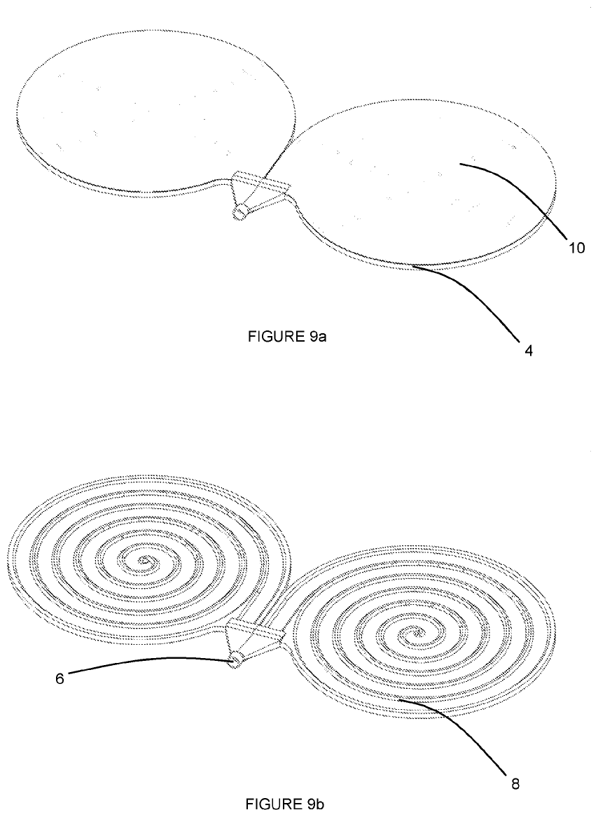

FIG. 9a is a perspective view of an alternative embodiment of the pad of the present invention; and

FIG. 9b is the pad of FIG. 9a, with the uppermost surface removed to show the path of a network of channels moulded into said pad and the use of a 2-way splitter valve.

A portable active cooling device 2 according to the present invention is shown in FIG. 1 of the accompanying drawings. The cooling device 2 comprises a selection of flexible pads 4 of various shapes and sizes designed to conform to various parts of a patient's body. For example, in FIG. 1, there is shown two pads 4 around the patient's thighs and a pad 4 around the patient's torso.

Each pad 4 comprises an inlet valve 6 in fluid communication with a network of channels 8.

The channels 8 are U-shaped in cross-section. i.e. the uppermost side is open, and the channels 8 are formed from a recess or groove cut into the surface of the pad 4. A thermally conductive hydrogel membrane 10 is glued across the top surface of the pad 4 to cover and seal the channel 8 and form a conduit 11 (shown most clearly in FIG. 7), wherein the walls of the conduit 11 comprise the pad 8 and the membrane 10. The channels 8 are moulded into the polyurethane pad 4. The cooling device 2 emulates IWI via use of the network of channels 8 and the thermally conductive hydrogel membrane 10 built into the pads 4 which adheres to the torso and upper legs. In an alternative embodiment, the recessed channel 8 is cut into the hydrogel membrane 10 and sealed by the surface of the pad 4 secured over it. It could also be envisaged that the channel 8 is made by forming a recess in both the pad 4 and the membrane 10. What is required is that the pad 4 and the membrane 10 form the walls of the channel 8. Forming open-sided channels 8 in the pad 4 covering said channels 8 by securing a flat sheet of thermally conductive membrane over the open side to provide a closed-sided conduit 11 through which gas can be injected represents a viable method of large scale manufacture of the apparatus 2.

As shown best in FIG. 2, a canister 14 of compressed gas coolant 12 is connected to the inlet valve and said gas 12 is injected into the pads 4 via said inlet valve 6. The gas 12 passes around the U-shaped channels 8 and is exposed to the hydrogel membrane 10 which forms the “lid” of said U-shaped channel 8. Rapid and sustainable cooling of the hydrogel membrane 10 then takes place. The coolant 12 is stored within lightweight, portable compressed gas canisters 14, creating a long-lasting modality and is able to be instantly activated, requiring no pre-preparation or specialist storage.

The channels 8 are moulded into the surface of the urethane pad 4 so that, in use, the injected gas flows directly over said pad 4. It is important that the surface finish of the pad 4 is not textured otherwise gas flow is inhibited and ice crystals, and subsequently cold spots, form. The pad 4 of the present invention comprises a self-skinning polyurethane flexible foam. During manufacture of the pad 4 of the present invention a back pressure is applied to the mould to enable the surface of the pad 4 to be significantly denser. This emulates similar properties to a typical elastomer whilst still retaining the weight of a foam. This provides a much smoother surface for the gas to travel over, significantly reducing ice crystal and therefore cold spot formation.

The gas canister 14, and indeed the cooling device 2 as a whole, is not reliant on electric power for functionality. The compressed canisters 14 can be stored indefinitely and provide cooling on demand. Prior art cooling garments such as those stated in US2020281284A1, US2019151141A1 and US2004064171A1 rely on either a battery pack or mains power, potentially limiting use in some scenarios.

The gas canisters 14 of the present invention hold hundreds of litres of gas under pressure in liquid form. Medical grade CO2 canisters hold substantial volumes of gas whilst remaining compact, lightweight and portable. For example, a canister containing 900 litres of CO2 weighs just 5 kg and is a viable and portable heat illness treatment option for use in the present invention.

The pads 4 do not interfere or restrict any vital monitoring or intervention. The chest of the patient is purposely left exposed to enable constant cardiac monitoring, external defibrillation and CPR to occur concurrently with cooling if required. This also accommodates for breast tissue and ensures that consistent skin adhesion is achieved. The pads 4 are also designed to be as visually intuitive as possible to provide guidance to the fitter in a stressful and time pressured scenario. The cooling device 2 of the present invention can be carried, applied and operated by a single person and can be used in emergency vehicles.

The pads 4 are made from lightweight, insulative material that adhere to the skin.

The body of the pad 4 is manufactured from flexible urethane foam. This material provides excellent thermal insulation, good conformability around the contours of the body, and is extremely lightweight. For example, a 250 mm×250 mm urethane pad prototype weighs just 31 g. The network of channels 8 moulded into the urethane pad 4 provides a path for the compressed gas 12 (CO2) from a connected gas canister 14 to flow through and enable effective dissipation throughout the entire pad 4. The path of the channels 8 (discussed in detail below) is such that as much of the hydrogel 10 contained in the membrane ‘lid’ is exposed to the gas as much as possible, whilst the pad 4 retains sufficient structural rigidity.

As is known, a hydrogel is a three-dimensional (3D) network of hydrophilic polymers that can swell in water and hold a large amount of water while maintaining the structure due to chemical or physical cross-linking of individual polymer chains.

The hydrogel membrane 10 of the present invention is produced in the form of a cured sheet. In another embodiment of the present invention hydrocolloids could be used rather than a cured sheet of hydrogel.

The use of hydrogel 10 as a thermal membrane in the present invention enables an excellent thermal conduction between the skin of the patient and the coolant gas. Critically, this enables higher cooling rates to be achieved, increasing the underlying efficacy of the technology. Additionally, the hydrogel 10 enables more efficient use of the coolant gas and increased temperature dissipation throughout the pad 4.

High strength flexible urethane adhesive is used to bond the pad 4 to the hydrogel membrane 10 to create a strong and durable chemical bond.

As gas is injected through the pad 4 at relatively high velocity, it is important to have a strong, durable and flexible bond between the pad 4 layer and the hydrogel membrane 10 layer to ensure there are no leakages while remaining able to form around the contours of the body.

The hydrogel membrane 10 of the present invention has a ‘dry top’, i.e a thin, flexible fabric mesh (not shown in the figures) that is bonded to the top/bottom surface on the hydrogel membrane 10 during manufacturing. The dry top enables a strong bond via an adhesive. A flexible urethane adhesive which bonds to the hydrogel membrane's ‘dry top’ creates a flexible, durable bond between the hydrogel membrane 10 and the pad 4.

Various embodiments of the path of the channels 8 in the pad 4 are shown in FIGS. 5, 6, 8b and 9b.

If the channels 8 change direction too abruptly, for example via acute angle turns, then solid carbon dioxide ice crystal formation (dry ice) takes place at these points within the channel 8. This is due primarily to changes in pressure and direction of the injected compressed gas 12. The channels 8 of the present invention have a minimal amount of acute angle turns, to minimise potential disruption of the gas flow. FIGS. 5 and 6 show an embodiment wherein the pattern of channels 8 is a 2×2 grid of squares formed by inwardly spiraling channels 8. FIGS. 8b and 9b show circular pads 4 in which the channels 8 spiral inwards to the centre.

As mentioned above, the channels 8 are fed with cool compressed gas 12 from the inlet valve 6, and, after passing over the hydrogel membrane 10, the gas 12 is released via one or more outlet valves 16. This ensures that there is no back-pressure stopping the injection of new cooling gas 12. Despite the low intrinsic heat capacity of the gas 12, as it is encouraged to flow continuously over the hydrogel membrane 10, it removes heat from the outer surface of the hydrogel, effectively cooling it.

The insulating foam outer layer of the pad 4 prevents the surrounding air (which is at ambient temperature) from counteracting the effect of the gas 12; that is, the only warming of the cool gas is from the hydrogel membrane 10 itself.

The gas 12 itself is stored in a pressure vessel (e.g. a gas canister) 14. This enables a large quantity of gas 12 at ambient pressure to be stored in a very small, uninsulated container at ambient temperature. By quickly expanding the compressed gas 12 out through a nozzle, it rapidly cools according to the Joule-Thomson effect; that is, as the gas 12 is expanded through a nozzle, the temperature drops. In this way, gas at a temperature below ambient can be injected into the channels 8 within the apparatus 2.

The Joule-Thomson Effect (or Joule-Kelvin Effect) is well understood and is routinely exploited in various refrigeration and cooling processes such as air conditioners, heat pumps, and liquefiers.

Expanding the compressed gas 12 out through a nozzle is done quickly enough that the process can be considered to be adiabatic (i.e. no heat energy enters or leaves the gas). If the gas 12 doesn't have to do any work when expanding (e.g. a free expansion into a vacuum), then the internal energy of the gas 12 can remain constant. In the simplest case, the internal energy of the gas 12 is equal to its “thermal kinetic energy” (which is measured by its temperature). So, if no work is done by the gas 12 when expanding, its temperature will remain constant.

However, the “internal energy” of a gas is not only composed of the gas's “thermal kinetic energy” (measured by its temperature) but also its “thermal potential energy”.

Again, if the gas 12 does no work when expanding (e.g. a free expansion into a vacuum), and therefore the internal energy of the gas 12 can remain constant, then any change to the gas's thermal potential energy must be balanced by a corresponding change in the thermal kinetic energy (and the gas's 12 temperature). In particular, as most gases expand, the thermal potential energy increases, and therefore the thermal kinetic energy (and therefore the temperature) will decrease.

Within this defined apparatus 2, the gas 12 in question does do work when expanding (e.g. pushing ambient air out of the way). The energy to perform this work must come from the “internal energy” of the gas 12. As noted above in the simplest case, if the internal energy drops (due to energy being extracted to perform work), then the “thermal kinetic energy” (and therefore the temperature) will also drop.

The overall effect on the gas 12 used in this apparatus is a combination of these two contributing effects, both of which lower the temperature of the gas 12 being expelled. Thus, the gas 12 used in this embodiment can be stored at high pressure and at ambient temperature, but when used becomes much colder than ambient very quickly.

The amount of cooling for any given pressure change (at various temperatures) has been well-established for a variety of gasses. This is termed the Joule-Thomson (or Joule-Kelvin) coefficient and is defined as the rate of change of temperature with respect to pressure P at constant enthalpy. The higher the value of the Joule-Thomson coefficient, the greater the amount of cooling.

Carbon Dioxide is well suited to use in this device as it is not only inexpensive and safe to store at high pressures, but it also possesses one of the highest Joule-Thomson coefficients of any commonly-available gas—for example, many times higher than Nitrogen, Oxygen, Hydrogen, Helium and Argon.

Accordingly, the cooling achieved from expanding Carbon Dioxide is significantly higher than for other gasses. The increased levels of cooling delivered by the use of Carbon Dioxide enables the cooling canisters to be smaller and lighter than compared to using the aforementioned alternative gases. Use of comparatively small and light gas canisters helps greatly with the portability of the apparatus.

It will be understood that many inwardly spiraling channel layouts are possible to cover various shapes of pad 4 with the goal always to minimise the number of acute turns in said channel 8 to minimise potential backflow.

With regard to the inlet valve 6, the design of this is such that cold spots do not form in the hydrogel around the valve 6 due to the narrow conduit in which, in use, very cold gas is travelling.

The present invention mitigates against the formation of cold spots through the use of a 2 and 4-way splitter valve 6.

The 4-way splitter valve 6 is shown best in FIGS. 5 and 6 and the 2-way splitter valve is shown best in FIG. 9b.

Use of splitter valves 6 enables the cooling effect to be dissipated to multiple parts of the pad 4 more effectively and significantly reduces the likelihood of cold spot formation around the valve 6.

The cooling device 2 of the present invention also comprises an outlet valve 16. The outlet valve 16 contains a soda lime filter for CO2 capture in order to minimise the risk of overexposure to CO2 of the patient and/or personnel administering the cooling device 2 in use.

The compressed gas coolant 12 injected into the pads 4 via the inlet valve 6 is compressed CO2. This provides substantial cooling capacity with minimal resources and enables the product to be portable and on-demand. The phase change from liquid to gas when releasing CO2 from a pressurised canister to the atmosphere draws heat from the environment. The temperature of the gas 12 within the pad 4 can be controlled via the flow rate from the canister 14. The high thermal conductance of the hydrogel 10 combined with the excellent insulative properties of the urethane pad 4 enables substantial cooling capacity with very low CO2 flow rate. Compressed CO2 canisters 14 are widely available and low cost providing a viable option for prototype testing and final product delivery. A series of different sized canisters can be selected according to scenario. For example, a static station located in a base medical centre could utilise a larger canister and supply, whereas dismounted operations would carry a smaller supply in compact, lightweight canisters 14.

The present invention is not limited to the specific embodiments described above. As discussed above, alternative paths of channel 8 which run through the pads 4 can be envisaged. The goal begin that all parts of the pad 4 are covered, the structural integrity of the pad 4 is not compromised, and the number of abrupt, acute angle bends in the channel 8 are minimised so as to minimise the possibility of cold spots forming and uneven cooling taking place throughout a pad 4.

The cooling apparatus itself can be made flexible and conformable to a subject by being formed from more than one discrete pads connected so that the pads may articulate with respect to each other.

The open-sided recess which makes the channel (8) in the surface of the pad may be formed using a stamp or press, for example the pad may be a pressed sheet and the open side of the channel may be covered with a substantially flat sheet of similar or the same material as the pressed sheet. The hydrogel membrane is then secured to the sheet to further cover the open side of the channel (8) in the surface of the pad (4). Alternatively, the pressed sheet may be covered with an opposing and complementary pressed sheet (i.e. one that is not substantially flat) such that a substantially tubular conduit is formed when the two sheets with channels are brought together. The channels are, for example, U-shaped in cross section to create a conduit with an O-shaped cross section. The pressed sheet which covers the channel (8) may simply be a mirrored version of the channel, or the cross-section geometry of the channel of the covering sheet may have a different cross section geometry to channel (8) in the pad (4).

1. A wearable cooling apparatus (2) comprising;
a pad (4), an inlet valve (6), a channel (8), and a thermally conductive membrane (10),
wherein the channel (8) is an open-sided recess formed in the surface of the pad (4) and the pad (4) is secured to the membrane (10) such that the open side of the channel (8) is covered by the membrane (10),
the covered channel (8) forms a conduit (11) arranged between the pad (4) and the membrane (10), wherein the walls of the conduit (11) comprise the surface of the pad (4) and the surface of the membrane (10),
and the inlet valve (6) is arranged in fluid communication with the conduit (11).

2. Apparatus according to claim 1, wherein a plurality of pads (4) are connected such that the pads (4) articulate with respect to each other.

3. Apparatus according to any of the preceding claims, wherein the pad is a pressed sheet.

4. Apparatus according to any of the preceding claims, wherein a sheet covers the open side of the channel (8) and is secured between the membrane (10) and the pad (4).

5. Apparatus according to claim 4 wherein the sheet that covers the open side of the channel (8) has a complementary channel formed in the sheet surface such that a conduit is created when said sheet is secured to the pad (4).

6. Apparatus according to any of claims 1 to 3, wherein the channel (8) is an open-sided recess formed in the surface of the membrane (4) and the pad (4) is secured adjacent to the membrane (10) such that the open portion of the channel (8) is covered by the adjacent pad (4).

7. Apparatus according to claim 1 or claim 3, wherein the channel (8) is an open-sided recess arranged in both the surface of the pad (4) and the membrane (10) such that the open portion of the channel (8) is covered by the adjacent pad (4) and the adjacent membrane (10).

8. Apparatus according to any of the preceding claims, wherein the membrane (10) comprises a material which enhances thermal conduction.

9. Apparatus according to any of the preceding claims, wherein the membrane (10) comprises a high water content chemical membrane.

10. Apparatus according to any of the preceding claims, wherein the membrane (10) comprises a hydrogel.

11. Apparatus according to any of the preceding claims, further comprising a canister (13) of compressed gas (12) in fluid communication with the inlet valve (6) and configured to inject compressed gas (12) into the conduit (11) via the inlet valve (6).

12. Apparatus according to claim 11, wherein the compressed gas (12) is carbon dioxide.

13. Apparatus according to claim 11 or 12, wherein the canister (14) is lightweight and portable.

14. Apparatus according to claims 11 to 13, wherein the canister (14) is configure to inject compressed gas (12) at high velocity.

15. Apparatus according to claims 11 to 14, wherein the temperature of the gas (12) injected into the conduit (11) is controlled via the flow rate of gas (12) from the canister (14).

16. Apparatus according to claims 11 to 15, wherein the gas canister (14) does not require electric power to release compressed gas (12).

17. Apparatus according to any of the preceding claims, wherein the channel (8) is U-shaped in cross section.

18. Apparatus according to any of the preceding claims, wherein the channel (8) is moulded into the surface of the pad (4) or the membrane (10).

19. Apparatus according to any of the preceding claims, wherein the channel (8) is in a spiral formation.

20. Apparatus according to claim 19, wherein the channel (8) spirals inwardly.

21. Apparatus according to any of the preceding claims, comprising a plurality of channels (8).

22. Apparatus according to claim 19 in combination with claim 21, wherein the spirals formed by the plurality of channels (8) are arranged in a grid.

23. Apparatus according to any of the preceding claims, wherein path of the channel (8) comprises no acute-angled bends.

24. Apparatus according to any of the preceding claims, wherein the pad (4) comprises polyurethane.

25. Apparatus according to any of the preceding claims, wherein the surface of the channel (8) is smooth.

26. Apparatus according to any of the preceding claims, wherein the pad (4) comprises flexible foam.

27. Apparatus according to claim 26, wherein the flexible foam comprises self-skinning polyurethane.

28. Apparatus according to any of the preceding claims, wherein the pad (4) is bonded to the membrane (10) via a high strength flexible urethane adhesive.

29. Apparatus according to any of the preceding claims, wherein the membrane (10) comprises a flexible mesh bonded to the surface positioned between the membrane (10) and the pad (4).

30. Apparatus according to any of the preceding claims, wherein the pad (4) comprises material that adheres to the skin.

31. Apparatus according to any of the preceding claims, wherein the pad (4) comprises insulative material.

32. Apparatus according to any of the preceding claims, wherein the membrane (10) is a cured sheet of hydrogel.

33. Apparatus according to any of the preceding claims, further comprising hydrocolloids arranged in the channel (8).

34. Apparatus according to any of the preceding claims, shaped so as to fit around a patient's torso.

35. Apparatus according to claim 34, further shaped so as to enable access to the chest of said patient.

36. Apparatus according to any of claims 1 to 33, shaped so as to fit around a patient's thigh.

37. Apparatus according to any of the preceding claims, wherein the inlet valve (6) is a 2-way splitter valve.

38. Apparatus according to any of the preceding claims, wherein the inlet valve (6) is a 4-way splitter valve.

39. Apparatus according to any of the preceding claims, further comprising an outlet valve (16).

40. Apparatus according to claim 39, wherein the outlet valve (16) contains a soda lime filter for carbon dioxide capture.

41. Apparatus according to any of the preceding claims, wherein the apparatus is portable.

42. A method of manufacturing the apparatus according to any of the preceding claims, comprising the steps of:
a) forming the pad (4) via a mould
b) applying a back pressure to the mould to enable the surface of the pad (4) to increase in density.

43. A method according to claim 42, further comprising the step of forming the channel (8) using a press.

44. A method of effecting the cosmetic physiological response of a wearer of the apparatus according to any of the preceding claims, the method comprising the steps of:
a) monitoring physiological aspects of the wearer
b) altering the cooling effect of the apparatus according to the preference of the wearer.

45. A method according to claim 44, wherein the physiological aspects include skin temperature, and/or skin colour, and/or clamminess of skin and/or amount of sweat produced.

46. A method according to claim 45, wherein the preference of the wearer is to increase the cooling effect of the apparatus when the monitored physiological aspects reach a defined threshold.

47. A method according to any of claims 44 to 46, wherein the cooling effect is altered based on whether the wearer is appearing red-faced, and/or hot, and/or with dry-skin.
